(19) United States
(12) Patent Application Publication
(10) Pub. No.: US 2010/0015718 A1
(43) Pub. Date: Jan. 21, 2010

(54) SUBSTRATE FOR ANALYZING COVERAGE OF SELF-ASSEMBLED MOLECULES AND ANALYZING METHOD USING THE SAME

(76) Inventors: Chil-Seong Ah, Daejon (KR); Ansoon Kim, Daejon (KR); Han-Young Yu, Daejon (KR); Chang-Geun Ahn, Daejon (KR); Jong-Heon Yang, Daejon (KR); In-Bok Baek, Chungbuk (KR); Chan-Woo Park, Daejon (KR); Seongjae Lee, Seoul (KR)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/517,769

(22) PCT Filed: Nov. 26, 2007
(86) PCT No.: PCT/KR07/05980

§ 371 Date: Jun. 4, 2009

(30) Foreign Application Priority Data
Dec. 6, 2006 (KR) 10-2006-0123294
Aug. 16, 2007 (KR) 10-2007-0082205

(51) Int. Cl. G01N 33/00

(52) U.S. Cl. 436/94

(57) ABSTRACT

Provided are a substrate for analyzing the coverage of self-assembled molecules and a method for analyzing the coverage of the self-assembled molecules in nanowire and nanochannel patterned on solid surface, solid surface, or bulk solid surface by using the nanoparticles. According to the method, the presence of specific functional groups of self-assembled molecules and the degree of reaction can be analyzed by introducing nanoparticles to a biomaterial immobilization substrate including self-assembled molecules and measuring the number of gold nanoparticles existing on the surface. The substrate for analyzing the coverage of self-assembled molecules includes: a biomaterial immobilization substrate; a self-assembled molecular layer formed on the substrate and having a functional group capable of reacting with an amine group; a capture DNA molecule having an amine group bounded to the self-assembled molecular layer; and a probe DNA molecule bound to the capture DNA molecule and having nanoparticles attached to on the surface.

### TECHNICAL FIELD

The present invention relates to a substrate for analyzing the coverage of self-assembled molecules and a method of analyzing the coverage of self-assembled molecules using the same; and more particularly, to a substrate for measuring the presence of specific functional groups of self-assembled molecules and the degree of reaction by introducing nanoparticles to a biomaterial immobilization substrate, and an analysis method using the same.

This work was supported by the IT R&D program of MIC/IITA [2006-S-007-01, “Ubiquitous Health Monitoring Module and System Development”].

### BACKGROUND ART

A biomaterial immobilization substrate is a device made of an existing semiconductor chip type by combining bio-organic matters isolated from creatures, such as enzymes, proteins, antibodies, DNA, microbes, animal and plant cells, animal and plant organs and neurons, with inorganic matters such as semiconductors. The biomaterial immobilization substrate can be largely classified into three: a DNA chip to which a DNA probe is immobilized; a protein chip to which a protein such as an enzyme, an antibody, or an antigen is immobilized; and a lab-on-a-chip on which pre-treating, biochemical reacting, detecting, and data-analyzing functions are integrated to impart an auto-analysis function.

To develop such a biomaterial immobilization substrate, the important thing is a technique for immobilizing a biomaterial in which an interface between the biomaterial and an immobilization substrate is efficiently formed, and the inherent functions of the biomaterial are fully utilized. Particularly, it is important that biomaterials are immobilized in a limited area of micrometer scale.

Representative methods of immobilizing biomaterials on the surface of a substrate include the Langmuir-Blodgett (LB) technique and the Self-Assembly (SA) technique. Among them, the LB technique employs the characteristic that amphiphilic molecules spread on the water surface are present in the form of monolayer films at the gas-liquid interface. The density of monolayers to be laminated on a solid surface can be adjusted by arbitrarily adjusting the density per area of the materials dispersed on the water surface, and a monolayer molecular film and a multilayer molecular film can be assembled on a solid substrate by adjusting the number of accumulation. However, such a fabrication process has the disadvantage that it requires much time and a complicated apparatus, and thus, the SA technique is more widely used than the above-described method.

Meanwhile, methods of measuring the presence of functional groups of self-assembled molecules or biomaterials or measuring the degree of reaction on a surface include FT-IR, XPS, fluorescence analysis, and so on. However, these methods have the drawbacks that the analysis procedure is complicated and they require a very specialized analysis technique. Particularly, the functional groups present on the surface of the self-assembled molecules are formed to be thin by very small organic molecules. This makes it difficult to determine the presence of functional groups, thus rendering it more difficult to find out the coverage state.

### DISCLOSURE

### Technical Problem

It is, therefore, an object of the present invention to provide a substrate for coverage analysis, which can measure the presence of functional groups present on the surface of self-assembled molecules and the degree of reaction by using nanoparticles without using complicated analysis equipment, and a method of analyzing the coverage of self-assembled molecules using the same.

Other objects and advantages of the present invention can be understood by the following description, and become apparent with reference to the embodiments of the present invention. Also, it is obvious to those skilled in the art of the present invention that the objects and advantages of the present invention can be realized by the means as claimed and combinations thereof.

### Technical Solution

In accordance with an aspect of the present invention, there is provided a substrate for analyzing coverage of self-assembled molecules, which includes: a biomaterial immobilization substrate; a self-assembled molecular layer formed on the biomaterial immobilization substrate and having a functional group capable of reacting with an amine group; a capture DNA molecule having an amine group bounded to the self-assembled molecular layer; and nanoparticles having a probe DNA molecule attached thereto and hybridized with the capture DNA molecule.

In accordance with another aspect of the present invention, there is provided a substrate for analyzing coverage of self-assembled molecules, which includes: a biomaterial immobilization substrate; a self-assembled molecular layer formed on the biomaterial immobilization substrate and having a functional group capable of reacting with an amine group; and nanoparticles having a capture DNA molecule attached thereto on the surface and bounded to the self-assembled molecular layer.

In accordance with another aspect of the present invention, there is provided a method for measuring coverage of self-assembled molecules, which includes the steps of: a) forming a self-assembled molecular layer on a biomaterial immobilization substrate by using molecules having a functional group capable of reacting with an amine group; b) binding the self-assembled molecular layer to a capture DNA molecule; c) complementarily hybridizing nanoparticles functionalized by the probe DNA with the capture DNA molecule; and d) measuring the number of the nanoparticles present on the surface of the biomaterial immobilization substrate.

In accordance with another aspect of the present invention, there is provided a method for measuring coverage of self-assembled molecules, which includes the steps of: a) forming a self-assembled molecular layer on a biomaterial immobilization substrate by using molecules having a functional group capable of reacting with an amine group; b) attaching a capture DNA molecule having an amine group to the surface of nanoparticles; c) binding the self-assembled molecular layer to the capture DNA molecule; and d) measuring the number of the nanoparticles present on the surface of the biomaterial immobilization substrate.

### ADVANTAGEOUS EFFECTS

In accordance with the present invention, it is possible to provide a substrate for coverage analysis, which can efficiently measure the presence of functional groups present on the surface of self-assembled molecules on the surface of the substrate and the degree of reaction by using nanoparticles without using complicated methods, such as FT-IR, XPS, and fluorescence method, and a method of analyzing the coverage of self-assembled molecules using the same.

### BRIEF DESCRIPTION OF THE DRAWINGS

FIG. 1 is a pattern diagram of a substrate for analyzing the coverage of a molecule having an aldehyde group in accordance with an embodiment of the present invention.

FIG. 2 is a view showing a process of self-assembling a molecule having an aldehyde group on the substrate in accordance with the embodiment of the present invention.

FIG. 3 is a view showing a process of introducing nanoparticles to a self-assembled molecule layer having an aldehyde group in accordance with the embodiment of the present invention.

FIG. 4 is a Field Emission Scanning Electron Microscope (FE-SEM) photograph of nanoparticles introduced to the surface of the substrate in accordance with the embodiment of the present invention.

FIGS. 5 to 7 are FE-SEM photographs of nanoparticles introduced to the surface of the substrate in accordance with the embodiment of the present invention.

### BEST MODE FOR THE INVENTION

The advantages, features and aspects of the invention will become apparent from the following description of the embodiments with reference to the accompanying drawings, which is set forth hereinafter, and thus, the invention will be readily carried out by those skilled in the art. Further, in the following description, well-known arts will not be described in detail if it seems that they could obscure the invention in unnecessary detail. Hereinafter, a preferred embodiment of the present invention will be set forth in detail with reference to the accompanying drawings.

A biomaterial immobilization substrate used in the present invention may be a transparent solid substrate or an opaque solid substrate such as silicon. Preferably, environmentally stable or chemical-resistant glass, polycarbonate, polyester, polyethylene (PE), polypropylene (PP), or a silicon wafer may be used for the substrate. However, the present invention is not limited to these materials.

The substrate may include a nanopattern, a nanoline, or a nanochannel on its surface, and may be surface-treated in order to improve reaction with self-assembled molecular layers. Surface treating agents used in the surface treatment may be, for example, a mercaptoethanol solution or mercaptopropionic acid solution, ethylene glycol, and polyethylene glycol.

The SA technique discussed above is frequently applied by using the so-called bottom-up method of assembling nano-size molecules into blocks while controlling their size and components and assembling them into a large structure having constant properties and functions. In order to fabricate a substrate for immobilizing biomaterials, such as proteins, DNA, enzymes, viruses, which is fabricated by the top-down method or bottom-up method, it is necessary to perform in advance the operation of immobilizing specific biomaterials, which are to be specifically bound to a target molecule, on the surface of the substrate to detect desired biomaterials.

Therefore, the substrate for analyzing the coverage of self-assembled molecules in accordance with the present invention forms, on the biomaterial immobilization substrate, a self-assembled molecular layer, having a functional group capable of reacting with an amine group, so that this molecular layer can be bound to the target molecule. One side of the self-assembled molecular layer has a functional group capable of reacting with the surface of the biomaterial immobilization substrate, and the other side thereof has a functional group capable of reacting with an amine group. Such a self-assembled molecular layer is self-assembled on the surface of the biomaterial immobilization substrate, thereby acting to immobilize biomaterials on the surface of the substrate.

The functional group capable of reacting with a functional group of the surface of the biomaterial immobilization substrate can be bound with the functional group of the surface of the substrate by a covalent bond or bound with a hydrophilic or hydrophobic functional group of the substrate by physicochemical adsorption. Examples thereof are functional groups, such as —SH, —NH₂, —Si(OCH₃)₃, —Si(OC₂H₅)₃, and —Si(Cl)₃. Further, the functional group capable of reacting with the amine group is not specifically limited so long as they can introduce DNA particles to the substrate by reaction with an amine group. Such an example may be an aldehyde group or carboxyl group.

Particularly, if the functional group of the surface of the substrate is a hydroxyl group (OH), the self-assembled molecules preferably have a trialkoxysilane functional group. Examples of such a compound include aminopropyl trimethoxysilane, aminopropyl triethoxysilane, aldehyde propyltrimethoxysilane, and aldehyde propyltriethoxysilane, but not limited thereto.

The substrate for coverage analysis in accordance with the present invention may further include a linker molecular layer formed between the biomaterial immobilization substrate and the self-assembled molecular layer so as to make the reaction therebetween more effective.

The linker molecular layer is formed on the biomaterial immobilization substrate. One side thereof has a functional group capable of reacting with the functional group of the surface of the biomaterial immobilization substrate, and the other side thereof has a functional group capable of reacting with the self-assembled molecular layer. Such a linker molecular layer is self-assembled on the surface of the substrate and used as medium for introducing subsequent self-assembled molecules, thereby acting to more efficiently immobilize biomaterials on the surface of the substrate.

The functional group capable of reacting with a functional group of the surface of the biomaterial immobilization substrate can be bound with the functional group of the surface of the substrate by a covalent bond or bound with a hydrophilic or hydrophobic functional group of the substrate by physicochemical adsorption. Examples thereof are functional groups, such as —SH, —NH₂, —Si(OCH₃)₃, —Si(OC₂H₅)₃, and —Si(Cl)₃. Particularly, if the functional group of the surface of the substrate is a hydroxyl group (OH), the linker molecules preferably have a trialkoxysilane functional group. Examples of such a compound include aminopropyl trimethoxysilane, aminopropyl triethoxysilane, and so on.

FIG. 1 illustrates a substrate for analyzing the coverage of a molecule having an aldehyde group in accordance with an embodiment of the present invention. Referring to this drawing, the substrate in accordance with the present invention will be described in more detail. The substrate of the present invention includes a linker molecular layer 101 formed on the surface of a biomaterial immobilization substrate 100, and a self-assembled molecular layer 102 having an aldehyde group formed on the top thereof.

As described above, the linker molecular layer 101 is formed as medium for more effectively achieving a reaction between the biomaterial immobilization substrate 100 and the self-assembled molecular layer 102. The substrate for analyzing the coverage of self-assembled molecules in accordance with the present invention can be formed by binding the self-assembled molecule layer directly to the biomaterial immobilization substrate without a linker molecular layer.

As for the coverage of a molecule having an aldehyde group, an introduced aldehyde group 103 and a capture DNA molecule 200 containing an amine group at the end form a chemical bond, and then a probe DNA molecule 201 bonded to the surface of nanoparticles is complementarily bonded to the capture DNA molecule 200, to thus guide the nanoparticles to the surface of the substrate. Analyzing the number of such nanoparticles can find out the coverage of the molecule having an aldehyde group bonded to the surface of the substrate. That is, the nanoparticles present on the surface of the substrate are caused by the molecule having an aldehyde group self-assembled and bonded to the surface of the substrate. This means that the more the number of the nanoparticles, the better the coverage of the self-assembled molecular layer having an aldehyde group on the surface of the substrate.

The substrate for analyzing the coverage of self-assembled molecules in accordance with the present invention can be fabricated by the following method, but not limited thereto.

The biomaterial immobilization substrate is washed with the aforementioned surface treating agent, and then the washed substrate is coated with a slurry coating solution containing many molecules to form the linker molecular layer. The material contained in the coating solution and used as a linker molecule is as described above, and the coating solution is prepared by adding the linker molecule to a dilution solvent.

The dilution solvent may be water, an organic solvent, or a mixed solvent of water and an organic solvent. The organic solvent is preferably selected from the group consisting of methanol, ethanol, propanol, a cellosolve solvent, and dimethylformaldehyde.

The concentration of the linker molecule contained in the coating solution is preferably 0.001 to 10 wt %. If the concentration is less than 0.01 wt %, the linker effect is not sufficient, whereas if it is more than 10 wt %, the coated substrate is not uniform.

A wet coating method may be used to coat the substrate with the linker molecular layer. Examples of wet coating methods include a self-assembly method, spin-coating, dipping, spraying, printing, and an LB technique. The self-assembly method is preferable in the sense that it can form a uniform molecular layer with a desired thickness. The linker molecular layer thus formed acts as medium for the binding the substrate and the molecular layer having an aldehyde group that is self-assembled.

The linker molecular layer can analyze a nanopattern, a nanoline, or a nanochannel on its surface. The length of the nanostructure is 1 to 50,000 nm, the height thereof is 1 to 5,000 nm, and the width thereof is 1 to 1,000 nm.

The substrate having the linker molecular layer formed thereon is dipped and reacted in a molecular solution having a functional group capable of reacting with a linker molecule at one end and a functional group capable of reacting with an amine group at the other end, to form a self-assembled molecular layer having a functional group capable of reacting with an amine group on the linker molecular layer.

The self-assembled molecular layer having a functional group capable of reacting with an amine group can analyze a nanopattern, a nanoline, or a nanochannel on its surface. The length of the nanostructure is 1 to 50,000 nm, the height thereof is 1 to 5,000 nm, and the width thereof is 1 to 1,000 nm.

The capture DNA has an amine group at one terminal end, and is bound to the self-assembled molecular layer by using this functional group. The binding is performed by chemical molecules, light, wet treatment, dry treatment, or a laser.

The nanoparticles can be introduced to the substrate by binding the capture DNA to the self-assembled molecular layer, and then complementarily binding the same to the probe DNA molecule attached to the surface of the nanoparticles. Alternatively, the nanoparticles may be introduced to the substrate by attaching the capture DNA molecule directly to the surface of the nanoparticles and then binding the same directly to the self-assembled molecular layer.

A method of attaching the capture DNA molecule or the probe DNA molecule to the nanoparticles is as follows.

The probe DNA molecule with a thiol molecule attached to one end (or the capture DNA molecule with a thiol molecule at one end and an amine functional group attached to the other end) is dipped in a gold nanoparticle solution to attach the DNA molecule to the surface of the nanoparticles.

The nanoparticles include at least one constituent selected from the group consisting of gold, silver, platinum, and copper. For analysis of the nanostructure, the diameter thereof is preferably 1 to 500 nm.

The capture DNA molecule having an amine group end is bounded to the substrate with the self-assembled molecular layer produced by the above-described method, and the probe DNA molecule attached to the surface of the nanoparticles is complementarily bounded to the capture DNA molecule, thus introducing nanoparticles to the biomaterial immobilization substrate. As a result, the number of the introduced nanoparticles is counted through an electron microscope, thereby analyzing the coverage of the self-assembled molecules.

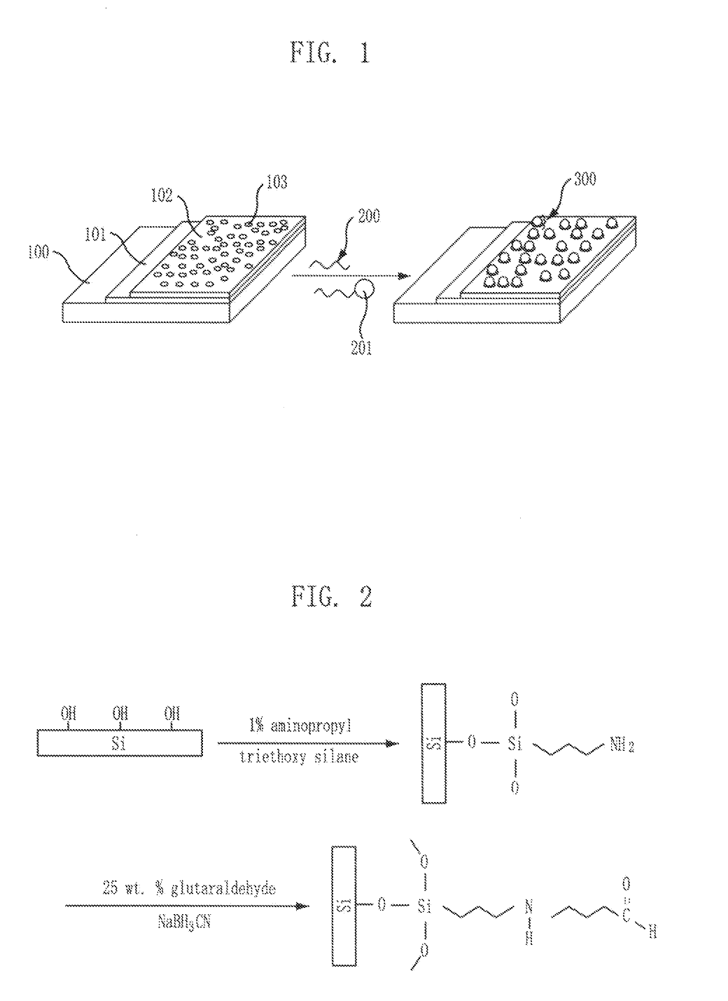

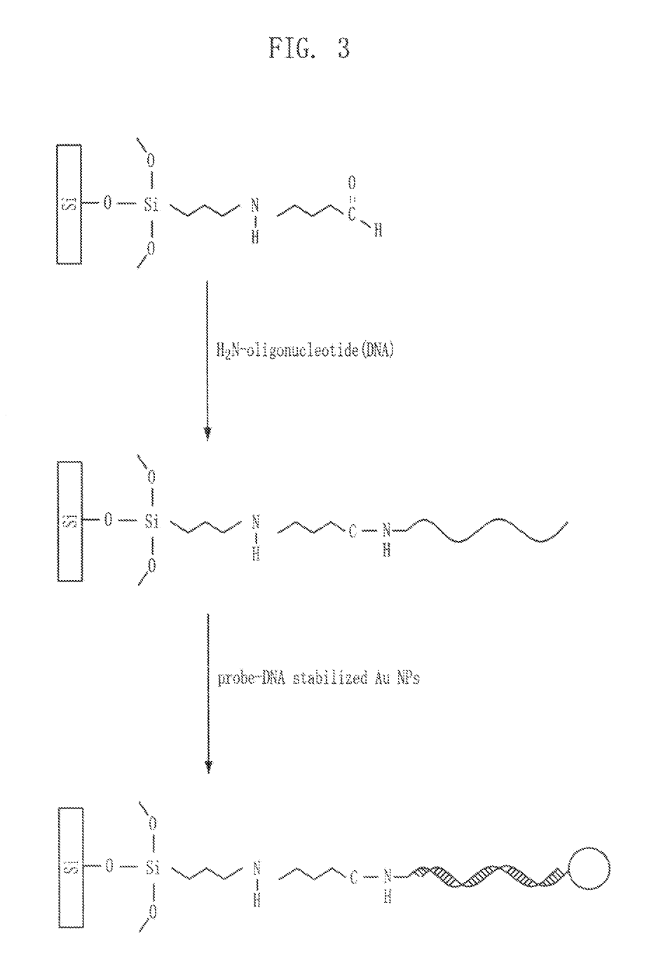

FIG. 2 illustrates a process of self-assembling an aldehyde group on the substrate in accordance with the embodiment of the present invention. FIG. 3 illustrates a process of binding nanoparticles having a DNA molecule to the molecule having a self-assembled aldehyde group to introduce the nanoparticles to the surface of the substrate in accordance with the embodiment of the present invention. Referring to FIGS. 2 and 3, aminopropyl triethoxysilane is immobilized on a biomaterial immobilization substrate having a hydroxyl group on the surface to introduce an amine group to the surface of the substrate, and then glutaraldehyude is reacted to finally introduce an aldehyde group to the surface of the substrate. Referring to FIG. 3, a capture DNA having an amine group is chemically bound to an end of the substrate to which an aldehyde group is introduced, and then nanoparticles stabilized by a probe DNA are complementarily bound to the capture DNA to introduce the nanoparticles to the surface of the substrate. In step of introducing nanoparticles to the surface of the substrate in FIG. 3, it is possible to apply the method of reacting nanoparticles stabilized by the DNA having an amine group at an end directly with a self-assembled aldehyde group on the surface of the substrate, as well as the method of chemically binding a capture DNA containing an amine group to an aldehyde group and then complementarily binding nanoparticles stabilized by a probe DNA to the capture DNA.

The substrate for analyzing coverage in accordance with the present invention may further include a linker molecular layer formed between the biomaterial immobilization substrate and the self-assembled molecular layer so as to make the reaction therebetween more effective.

In step of measuring the number of nanoparticles, analysis is done with naked eye observation through the use of measuring equipment, such as an FE-SEM, an optical microscope, an AFM, a TEM, or the like, which allows a user to see the shape of the nanoparticles.

Hereinafter, the present invention will be descried in more detail through embodiments, but the scope of this invention is not limited to these embodiments.

### EXAMPLE

A substrate for analyzing the coverage of self-assembled molecules in accordance with the present invention was fabricated in the following method.

As shown in FIG. 1, an aldehyde functional group was introduced by using a silicon substrate Si. First, O₂ plasma ashing was done to the silicon substrate Si for 5 minutes at 25 W to introduce a hydroxyl functional group. Next, the silicon substrate was dipped in 20 ml of an ethanol solution of 1% APTES (aminopropyl triethoxy silane) for 30 minutes, and then baked at 120° C., to thus introduce an amine functional group. Finally, the substrate was dipped in a 25 wt % glutaraldehyde solution having 0.1 g of NaBH₃CN for two hours to introduce an aldehyde functional group.

FIG. 3 shows a process of attaching DNA-gold nanoparticles to a silicon substrate with an aldehyde group introduced thereto. First, a DNA molecule having an amine functional group at one end was dipped in 4 mM of an NaBH₃CN reducing agent solution (pH 8.4) and reacted for 6 hours, to thus introduce the DNA molecule to the silicon substrate. Next, gold nanoparticles with the DNA molecule immobilized on the surface and the silicon substrate on which the DNA molecule is immobilized were dipped in a DNA solution, and then complementarily bounded and reacted with each other for 4 hours, to thus introduce gold nanoparticles to the silicon substrate.

### EXPERIMENTAL EXAMPLE

### Measurement of Number of Nanoparticles Bound to Substrate Surface

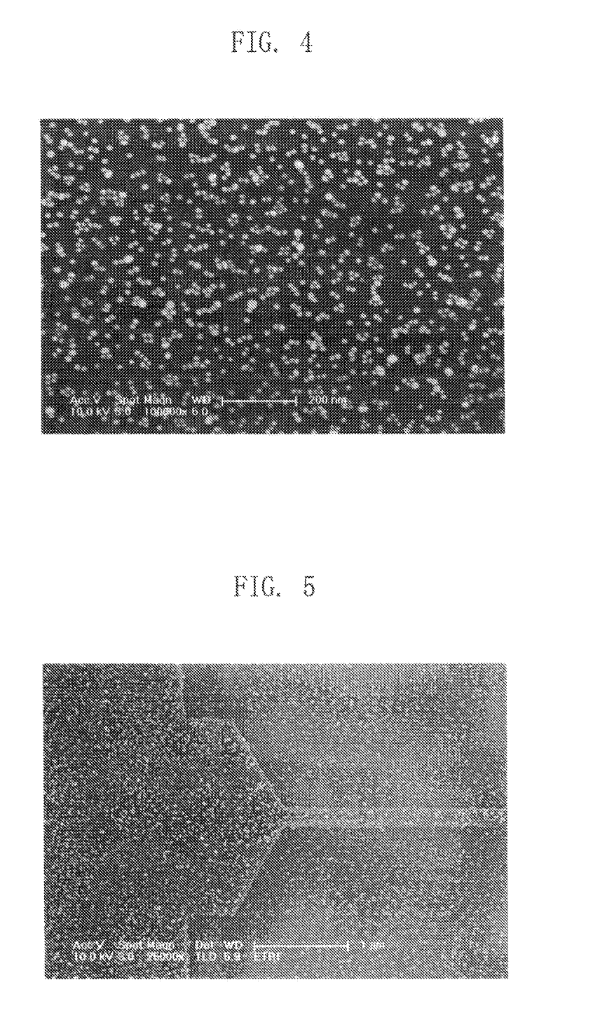

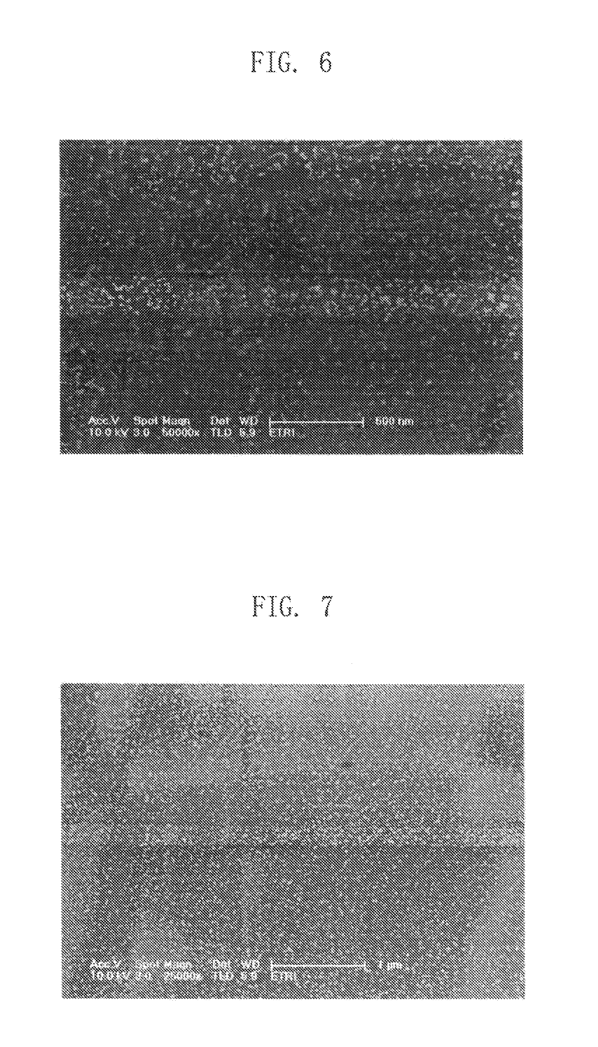

The number of nanoparticles bound to the surface of the substrate fabricated through the foregoing embodiment was measured by using an FE-SEM, and the results thereof were shown in FIGS. 4 and 5 to 7.

FIG. 4 shows an FE-SEM image of nanoparticles that are introduced to a bulk silicon surface by chemically bounding and reacting a capture DNA containing an amine group produced in the foregoing embodiment with an aldehyde group present on the surface of a substrate and then complementarily binding nanoparticles stabilized by a probe DNA to the capture DNA. The nanoparticles have a diameter of 13 nm, and have a very good coverage of about 1,100 nanoparticles/μm². The nanoparticles on the surface of the substrate are caused by the aldehyde group on the surface of the substrate, and thus it can be said that the coverage of the nanoparticles can replace the coverage of the aldehyde group present on a solid surface. Although the nanoparticles used have a diameter of 13 nm, since a DNA of 12 bases in length, which is equal to approximately 3 nm, is present on the surface of the nanoparticles, the surface area occupied by one nanoparticle can be estimated to have a diameter of approximately 19 nm. This means that there exists the coverage of at least 50% of self-assembled molecules containing aldehyde group on the surface of the substrate.

FIGS. 5 to 7 show an FE-SEM image of nanoparticles that are introduced to silicon nanolines fabricated on the surface of a silicon oxide substrate by chemically bounding and reacting a capture DNA containing an amine group produced by the foregoing embodiment with an aldehyde group present on the surface of a substrate and then complementarily binding nanoparticles stabilized by a probe DNA to the capture DNA. The coverage of a self-assembled molecular layer containing an aldehyde group in the nanolines patterned on the surface of the substrate can be known by checking the number of the nanoparticles. It can be seen that a large number of nanoparticles are present on the nanolines (1,100 nanoparticles/μm²).

The present application contains subject matter related to Korean Patent Application Nos. 2006-0123294 and 2007-0082205, filed in the Korean Intellectual Property Office on Dec. 6, 2006, and Aug. 16, 2007, the entire contents of which is incorporated herein by reference.

While the present invention has been described with respect to the specific embodiments, it will be apparent to those skilled in the art that various changes and modifications may be made without departing from the spirit and scope of the invention as defined in the following claims.

What is claimed is:

1. A substrate for analyzing coverage of self-assembled molecules, comprising:
a biomaterial immobilization substrate;
a self-assembled molecular layer formed on the biomaterial immobilization substrate and having a functional group capable of reacting with an amine group;
a capture DNA molecule having an amine group bounded to the self-assembled molecular layer; and
nanoparticles having a probe DNA molecule attached thereto and hybridized with the capture DNA molecule.

2. The substrate of claim 1, wherein the biomaterial immobilization substrate is at least one selected from the group consisting of glass, silicon-on-insulator (SOI), silicon, TiO₂, silicon oxide, polycarbonate, polyester, polyethylene, polypropylene, and a wafer.

3. The substrate of claim 1, wherein one side of the self-assembled molecular layer has a functional group capable of reacting with the surface of the biomaterial immobilization substrate, and the other side thereof has a functional group capable of reacting with an amine group.

4. The substrate of claim 3, wherein the functional group capable of reacting with the functional group of the surface of the substrate is at least one selected from the group consisting of —SH, —NH₂, —Si(OCH₃)₃, —Si(OC₂H₅)₃, and —Si (Cl)₃.

5. The substrate of claim 1, further comprising a linker molecular layer.

6. The substrate of claim 5, wherein one side of the linker molecular layer has a functional group capable of reacting with the functional group of the surface of the biomaterial immobilization substrate, and the other side thereof has a functional group capable of reacting with the self-assembled molecular layer

7. The substrate of claim 7, wherein the functional group capable of reacting with the functional group of the surface of the substrate is at least one selected from the group consisting of —SH, —NH₂, —Si(OCH₃)₃, —Si(OC₂H₅)₃, and —Si(Cl)₃.

8. A substrate for analyzing coverage of self-assembled molecules, comprising:
a biomaterial immobilization substrate;
a self-assembled molecular layer formed on the biomaterial immobilization substrate and having a functional group capable of reacting with an amine group; and
nanoparticles having a capture DNA molecule attached thereto on surface and bounded to the self-assembled molecular layer.

9. The substrate of claim 8, wherein the biomaterial immobilization substrate is at least one selected from the group consisting of glass, silicon-on-insulator (SOI), silicon, TiO₂, silicon oxide, polycarbonate, polyester, polyethylene, polypropylene, and a wafer.

10. The substrate of claim 8, wherein one side of the self-assembled molecular layer has a functional group capable of reacting with the surface of the biomaterial immobilization substrate, and the other side thereof has a functional group capable of reacting with an amine group.

11. The substrate of claim 10, wherein the functional group capable of reacting with the functional group of the surface of the substrate is at least one selected from the group consisting of —SH, —NH₂, —Si(OCH₃)₃, —Si(OC₂H₅)₃, and —Si(Cl)₃.

12. The substrate of claim 8, further comprising a linker molecular layer.

13. The substrate of claim 12, wherein one side of the linker molecular layer has a functional group capable of reacting with the functional group of the surface of the biomaterial immobilization substrate, and the other side thereof has a functional group capable of reacting with the self-assembled molecular layer.

14. The substrate of claim 13, wherein the functional group capable of reacting with the functional group of the surface of the substrate is at least one selected from the group consisting of —SH, —NH₂, —Si(OCH₃)₃, —Si(OC₂H₅)₃, and —Si(Cl)₃.

15. A method for measuring coverage of self-assembled molecules, comprising the steps of:
a) forming a self-assembled molecular layer on a biomaterial immobilization substrate by using molecules having a functional group capable of reacting with an amine group;
b) binding the self-assembled molecular layer to a capture DNA molecule;
c) complementarily hybridizing nanoparticles functionalized by the probe DNA with the capture DNA molecule; and
d) measuring the number of the nanoparticles present on the surface of the biomaterial immobilization substrate.

16. The method of claim 15, wherein, in the step d) of measuring the number of the nanoparticles, analysis is done with naked eye observation using measuring equipment.

17. The method of claim 16, wherein the measuring equipment is at least one selected from the group consisting of an FE-SEM, an optical microscope, an AFM, and a TEM.

18. A method for measuring coverage of self-assembled molecules, comprising the steps of:
a) forming a self-assembled molecular layer on a biomaterial immobilization substrate by using molecules having a functional group capable of reacting with an amine group;
b) attaching a capture DNA molecule having an amine group to the surface of nanoparticles;
c) hybridizing the self-assembled molecular layer with the nanoparticles having the capture DNA molecule attached thereto; and
d) measuring the number of the nanoparticles present on the surface of the biomaterial immobilization substrate.

19. The method of claim 18, wherein, in the step d) of measuring the number of the nanoparticles, analysis is done with naked eye observation using measuring equipment.

20. The method of claim 19, wherein the measuring equipment is at least one selected from the group consisting of an FE-SEM, an optical microscope, an AFM, and a TEM.
